(19) United States
(12) Patent Application Publication
(10) Pub. No.: US 2011/0189309 A1
(43) Pub. Date: Aug. 4, 2011

(54) TOPICAL MEDICAMENT

(75) Inventor: Peter O'Gorman, Ferny Grove (AU)

(73) Assignee: WHIPBIRD PAIN RELIEF PTY LTD ACN 132 827 411

(21) Appl. No.: 13/003,715

(22) PCT Filed: Jul. 11, 2008
(86) PCT No.: PCT/AU2008/001019

§ 371 Date: Apr. 15, 2011

(30) Foreign Application Priority Data
Jul. 12, 2007 (AU) 2007903790

(51) Int. Cl. A61K 33/14; A61P 29/00; A61P 31/00

(52) U.S. Cl. 424/681

(57) ABSTRACT

The present invention relates, in general, to topical medicinal formulations that provide pain relief and enhance recovery from injury. In particular, the invention relates to a topical medicinal formulation comprising 0.35 to 35% of a soluble salt of a biologically acceptable alkali metal or alkaline earth metal such as sodium, potassium, lithium, magnesium and calcium and their use in methods of treating pain or enhancing healing.

### FIELD OF THE INVENTION

The present invention relates, in general, to topical medicinal formulations that provide pain relief and enhance recovery from injury. In particular, the invention relates to a topical medicinal formulation comprising 0.35 to 35% of a soluble salt of a biologically acceptable alkali metal or alkaline earth metal such as sodium, potassium, lithium, magnesium and calcium and their use in methods of treating pain or enhancing healing.

### BACKGROUND OF THE INVENTION

There are many topical treatments for pain or to enhance healing of an injury or disease. Some treatments are available “over the counter” at pharmacies and/or supermarkets and others are prescription only medicines. Many products available include expensive ingredients, require regular and frequent application and provide relief of pain for only short periods. Many products that provide pain relief do not enhance recovery from injury. There is a need for further pain relieving formulations or formulations that enhance recovery from injury.

### SUMMARY OF THE INVENTION

The present invention is predicated, at least in part, on the discovery that a product containing 0.35 to 35% of at least one soluble salt provides pain relief and enhances recovery from injury. Without wishing to be bound by theory, it is believed that the product provides pain relief and enhances recovery by reinvigorating the body's electrical circuits that have been blocked or suppressed by damaged or unhealthy cells caused by injury or disease.

In one aspect of the present invention there is provided a topical medicinal formulation comprising 0.35 to 35% of at least one soluble salt of a biologically acceptable alkali metal or alkaline earth metal and a pharmaceutically acceptable carrier, wherein the formulation is in the form of a cream, lotion, ointment, liniment, gel, poultice, cold or hot compress, dressing or plaster cast.

In some embodiments, the amount of salt is 0.5 to 20%, especially 1 to 10%, more especially 3 to 10% and most especially about 5.0 to 10%, for example, about 5.9% or 8.2%, or 9.0 to 9.3%.

In some embodiments, the topical medicinal formulation is in the form of a cream, lotion, ointment, liniment or gel. In other embodiments, the formulation is applied as part of or impregnated within a dressing that is applied to an injury, such as a bandage, poultice, cold or hot compress or a plaster cast.

In another aspect, there is provided a method of treating or preventing pain comprising applying to the skin of a subject a topical medicinal formulation comprising 0.35 to 35% of at least one soluble salt of a biologically acceptable alkali metal or alkaline earth metal and a pharmaceutically acceptable carrier, wherein the formulation is in the form of a cream, lotion, ointment, liniment, gel, poultice, cold or hot compress, dressing or plaster cast.

In yet another aspect of the present invention, there is provided a method of enhancing healing of an injury in a subject comprising applying to the skin of the subject in the proximity of the injury a topical medicinal formulation comprising 0.35 to 35% of at least one soluble salt of a biologically acceptable alkali metal or alkaline earth metal and a pharmaceutically acceptable carrier, wherein the formulation is in the form of a cream, lotion, ointment, liniment, gel, poultice, cold or hot compress, dressing or plaster cast.

In some embodiments the subject is a human. In other embodiments, the subject is a racing animal such as a horse or dog, especially a horse.

In yet another aspect of the present invention there is provided a method of relieving sore ore tired muscles comprising applying to the skin of a subject a topical medicinal formulation comprising 0.35 to 35% of at least one soluble salt of a biologically acceptable alkali metal or alkaline earth metal and a pharmaceutically acceptable carrier, wherein the formulation is in the form of a cream, lotion, ointment, liniment, gel, poultice, cold or hot compress, dressing or plaster cast.

In some embodiments, the sore or tired muscles result from a workout or strenuous or unusual physical exercise or activity.

### DETAILED DESCRIPTION OF THE INVENTION

The topical medicinal formulation of the invention comprises 0.35% to 35% of at least one soluble salt of a biologically acceptable alkali metal or alkaline earth metal. As used herein, the term “biologically acceptable alkali metal or alkaline earth metal” refers to alkali metals and alkaline earth metals that will not disturb homeostatic balance in the body being treated. Examples of suitable alkali metal or alkaline earth metals for use in salts of the invention are selected from sodium, potassium, lithium, magnesium and calcium. In some embodiments, the at least one soluble salt is a salt of sodium, potassium or lithium, especially sodium or potassium and most especially sodium. In some embodiments, the salt is sea salt. Sea salt is obtained by evaporating sea water and contains predominantly sodium chloride but also includes many trace elements and trace amounts of other salts such as potassium, calcium and magnesium salts as well as sulfates. In some embodiments, the sea salt is fine grade, such as Pacific fine sea salt. In some embodiments, the sea salt is bacteriostatic.

In some embodiments, the at least one soluble salt is a halide salt such as a chloride, bromide or iodide or a sulfate, carbonate or phosphate salt. In some embodiments, the salt is a chloride salt.

In some embodiments, the soluble salt is sodium chloride. In some embodiments, the at least one soluble salt is sea salt, such as Pacific fine sea salt. In some embodiments more than one soluble salt is included. For example, in some embodiments, the at least one soluble salt includes sodium chloride and magnesium chloride. In some embodiments the at least one soluble salt is sea salt and magnesium chloride.

In some embodiments, the formulation may include further optionally additional trace elements found in the ocean. Such trace elements may be found in products such as ocean minerals, which are concentrates of minerals found naturally in the ocean. Ocean minerals are obtained by concentrating sea salt and removing sodium salts. The predominant salt in ocean minerals is magnesium sulfate but other trace elements are present

As used herein, the term “soluble salt” refers to a salt that is soluble in or may be at least partially dissolved in the formulation. In some embodiments, the salt is completely soluble in the formulation.

The amount of soluble salt present in the topical medicinal formulation is between 0.35 to 35% weight of salt per volume of the formulation (w/v). In some embodiments, the salt is present in an amount of 0.5 to 20%, 1 to 15%, 1 to 10%, 3 to 10% or about 5.0 to 8.5% (w/v). In one embodiment, the at least one salt is included in the composition in an amount of about 5.9% (w/v). In another embodiment the at least one salt is included in the composition in an amount of about 8.2%. In yet another embodiment, the at least one salt is present in an amount of 9.0 to 9.3%. In some embodiments more than one salt is present.

In some embodiments, the topical medicinal formulation is in the form of a cream, lotion, ointment, liniment or soft gel.

The medicinal formulation may also include optional components that also have healing properties, such as bioavailable oxygen, copper, glucosamine, sources of sulphur and extracts from plants.

Copper may be added in a homeopathic form such as cuprum. Copper is required by many enzymes in the body.

Glucosamine (C₆H₁₃NO₅) is an amino sugar that is used biologically in the synthesis of glycosylated proteins and lipids. It is a commonly used supplement for maintenance and rebuilding of cartilage in the body and can be used in the treatment of osteoarthritis. Glucosamine may be present in the formulation in an amount from 0.01% to 5% by weight of the composition, especially 0.05% to 3%, more especially 0.1% to 3%. In some embodiments, the glucosamine may be present in about 0.2% by weight of the composition. In other embodiments, the glucosamine may be present in an amount of about 2% by weight of the composition.

Sulfur is required in the body in essential amino acids such as cysteine and methionine and is important in the synthesis and maintenance of collagen and keratin. One source of sulphur is methyl sulfonyl methane (MSM, (CH₃)₂SO₂). In some embodiments, the sulphur source, such as MSM is added to the composition in an amount of 1 g to 10 g per litre of composition (w/v), especially 1 g to 5 g per litre, more especially 3 g per litre.

Bioavailable oxygen may be present in the composition in an amount of 20 to 30% by weight, especially about 25%.

Plant extracts may impart not only pleasant odour but also medicinal properties useful in healing wounds or muscular injuries, such as anti-inflammatory properties, antibiotic properties, deodorising properties or muscle relaxing properties. Suitable plant extracts include Aloe Vera juice, Boswellia extract, Eucalyptus oil, Arnica extract, Lantana Camara extract, Hypericum (St John's wort) extract, methyl salicylate oil and/or oil of wintergreen, clove bud oil, peppermint oil, and black pepper oil. Together with the cuprum, the plant extracts are added to the composition. The cuprum and plant extracts are generally present in the composition in an amount of from 1% to 10% by weight (w/w), especially 3% to 8% by weight, more especially about 5% to 8%.

The medicinal formulation may also include antibiotic and/or anti-inflammatory compounds that are not from natural extracts, for example, it may include the anti-inflammatory compound, ibuprofen or other non-steroidal anti-inflammatory drugs.

As used herein, the term “cream” refers to a topical water-soluble preparation that is for application to the skin.

As used herein, the term “lotion” refers to a low to medium viscosity topical preparation for application to skin that is an oil-in-water or water-in-oil emulsion.

An “ointment” as referred to herein is a viscous semi-solid preparation for topical application to the skin.

As used herein, the term “liniment” refers to a topical preparation formulated with a solvent that evaporates quickly in air at body temperature that is for application to the skin with friction, such as rubbing.

A “soft gel” as used herein refers to a low viscosity or semi-solid colloidal composition of interconnected particles in a liquid.

In some embodiments, the medicinal formulations are oil-in-water emulsions or biphasic systems comprising greater than 50% water and/or water miscible solvent such as ethanol.

The medicinal formulations of the invention may further contain components normally used in the preparation of creams, lotions, ointments, liniments and gels, including emollients or softening agents, emulsifying or thickening agents, humectants and/or moisturisers, gelling agents, preservatives, oils, waxes, solvents, fragrances, dyes, antioxidants, antifoaming agents, stabilising agents, pH adjusters and the like.

Suitable oily phases include hydrocarbons such as soft white paraffin, liquid paraffin, mineral oils and the like. Typically, the oily phase will be present in the composition in an amount of about 0.5 to about 20% by weight of the composition, especially about 1 to 15% by weight.

Suitable emulsifiers or thickeners are those normally used in the preparation of creams, lotions or ointments. Exemplary emulsifiers and thickeners include ethers of polyethylene glycol and fatty alcohols, cetyl alcohol, stearyl alcohol, sorbitol and other non-ionic emulsifying waxes, polyoxyethylene stearyl or cetyl alcohol ethers, glyceryl monostearate, polyoxyethylene sorbitan palmitate, Tween 20, 21, 40, 60, 65, 80, 81 or 85, polyoxyethylene glycol ethers of fatty alcohols such as cetearyl alcohol (Ceteareth-20), monoglycerides and fatty alcohols, fatty acid esters of alcohols having 3-21 carbon atoms, such as glyceryl monostearate and glyceryl monopalmitate.

Typically emulsifier-thickeners are present in an amount of 2 to 15% by weight of the formulation, especially 2 to 12% by weight.

Suitable emollients or softening agents include cetyl esters, wax and natural spermaceti wax, petrolatum, glyceryl monooleate, myristyl alcohol and isopropyl palmitate. Typically, emollients are present in an amount of up to 10% by weight of the composition.

A suitable humectant or moisturiser is glycerin, which may also be considered an emollient. Typically glycerin is present in the formulation up to about 20% by weight, for example about 2 to about 12% by weight.

Preservatives such as methyl paraben, propyl paraben, quaternium-15 and chlorocresol may be present. Other preservatives having anti-microbial activity, such as anti-fungal or antibacterial activity, may be present. An example is DMDM Hydantoin. Typically preservatives are present in an amount of up to 0.5% by weight of the composition.

Suitable pH adjusters are known in the art. For example, triethanolamine may be incorporated in the composition as a pH adjuster. Triethanolamine may also provide emulsifying activity.

Suitable waxes that may be included are known in the art. An example of a suitable wax is stearic acid.

Suitable solvents include any solvent that is pharmaceutically approved for topical application. Examples include water and ethanol.

Suitable gelling agents that may be used in the formulations of the invention include, but are not limited to, polysaccharides and gums. For example, carboxymethylcellulose, alginic acid, agar, xanthan gum, gum arabic and the like.

Antioxidants may also be included in the formulations of the invention, particularly those good for the skin, for example, vitamin E (tocopherol). In some embodiments, tocopherol salts such as tocopherol acetate are included in the composition.

A formulation of the invention may be prepared by dissolving the salt in the aqueous phase of the formulation then preparing the cream, lotion, ointment, liniment or gel by methods known in the art.

Alternatively, the salt can be blended with and dissolved in a pre-prepared cream, lotion, ointment, liniment or gel composition. For example, in one embodiment the salt may be blended into and dissolved in sorbolene cream, such as HB sorbolene or vegetable sorbolene, optionally together with other components of the formulation.

In another aspect of the invention, the topical medical formulation is in the form of a poultice, hot or cold compress, dressing or a plaster cast or splint that is for use in direct contact with the skin. For example, the salt may be added to a plaster composition containing gypsum or plaster of Paris (calcined calcium sulfate) and then formed into a plaster cast in the normal manner.

As used herein, the term “poultice” refers to a soft moist mass, often heated and medicated, which may be spread on a cloth and placed over the skin or placed directly on the skin to treat an aching, inflamed or painful part of the body. For example, a carrier such as bran, oats, slippery elm powder and the like can be boiled to a soft mass and mixed with at least one salt according to the invention and applied to the skin.

Hot or cold compress compositions are used to apply heat or cold to an injury or pain. In its simplest form, a cloth or bandage soaked in hot water or cold containing at least one salt according to the invention is wrung out and placed on the site of pain or injury. Optionally the composition may include essential oils. Other hot and cold compresses may include compositions that retain heat or cold and can be heated in a microwave or cooled in a freezer.

A dressing as used herein, includes a cream, lotion, ointment or gel formulation of the invention incorporated into a transdermal patch covering that may be applied directly to the skin and expose the formulation to the skin. The patch dressing may be self adhesive and may be optionally sterile.

The topical medicinal formulation of the invention is useful in the treatment or prevention of pain, for enhancing recovery or healing of an injury and for the treatment of or amelioration of nerve pain that is able to be topically treated.

Accordingly, in one aspect of the invention there is provided a method of treating or preventing pain comprising applying to the skin of a subject a topical medicinal formulation comprising 0.35 to 35% of at least one soluble salt of a biologically acceptable alkali metal or alkaline earth metal and a pharmaceutically acceptable carrier, wherein the formulation is in the form of a cream, lotion, ointment, liniment, gel, poultice, cold or hot compress, dressing or plaster cast.

In another aspect of the invention there is provided a method of enhancing healing of an injury in a subject comprising applying to the skin of the subject in the proximity of the injury a topical medicinal formulation comprising 0.35 to 35% of at least one soluble salt of a biologically acceptable alkali metal or alkaline earth metal and a pharmaceutically acceptable carrier, wherein the formulation is in the form of a cream, lotion, ointment, liniment, gel, poultice, cold or hot compress, dressing or plaster cast.

In yet another aspect of the invention, there is provided a method of relieving sore or tired muscles in a subject a topical medicinal formulation comprising 0.35 to 35% of at least one soluble salt of a biologically acceptable alkali metal or alkaline earth metal and a pharmaceutically acceptable carrier, wherein the formulation is in the form of a cream, lotion, ointment, liniment, gel, poultice, cold or hot compress, dressing or plaster cast.

The topical medical formulations are suitable for treating mammals. The term “mammal” as used herein includes humans, racing animals (eg. horses or dogs), primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). In some embodiments, the mammal is human or a laboratory test animal, especially a human.

An “effective amount” means an amount necessary at least partly to attain the desired response, or to delay the onset or inhibit progression or halt altogether, the onset or progression of a particular condition being treated. The amount varies depending upon the health and physical condition of the individual to be treated, the taxonomic group of individual to be treated, the hairiness of the individual to be treated, the degree of protection desired, the formulation of the composition, the assessment of the medical situation, and other relevant factors. It is expected that the amount will fall in a relatively broad range that can be determined through routine trials. An effective amount when administered to hairless skin is about 1 mL of formulation containing about 5.9% salt to cover about 800 to 950 square cm of skin, especially about 875 sq cm. An effective amount when administered to skin with hair cover is about 1 mL of formulation containing about 5.9% salt to cover about 400 to 560 sq cm of skin, especially about 480 sq cm. Dosages may increase if the formulation is used to massage an area affected by pain and/or injury. Dosage regimes may be adjusted to provide the optimum therapeutic response. For example, several divided doses may be administered daily, weekly, monthly or other suitable time intervals, or the dose may be proportionally reduced as indicated by the exigencies of the situation.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

The methods of the invention may be used to treat pain that is acute or chronic, especially musculoskeletal pain or pain caused by trauma, surgery or skin breakage. The methods of the invention are also suitable for treatment of pain associated with headaches or migraines. For example, the methods of the present invention are suitable for treating or preventing pain associated with arthritis, especially osteoarthritis, tenderness of or injury to ligaments, muscles, nerves, joints, cartilage or spinal discs including tendonitis, torn ligaments, sprains, strains, carpel tunnel syndrome, rotator cuff tendonitis, tension neck syndrome, bruising, back ache, bone breaks, canal stenosis, tennis elbow or pain associated with headaches or migraines. The methods of the invention may also be suitable for treating ulcers, such as leg ulcers, and/or pain associated with ulcers.

The methods of the invention are also able to treat or ameliorate nerve pain, neuralgia or neuropathy, particularly neuralgia and neuropathy that is able to be treated by topical application. Nerve pain can result from mechanical injury to the nerve, nerve degeneration, inflammation, nerve compression, infection, or chemical exposure. Peripheral neuropathies often result in numbness, tingling, prickling sensations, sensitivity to touch and burning pain. Such symptoms may be ameliorated or relieved by the methods of the present invention. Neuralgias often result in specific pain on or near the surface of the body in a specific location and include pain along a specific nerve, such as the sciatic nerve, sharp stabbing pain, constant burning pain, increased sensitivity of skin or numbness. Such nerve pain may be sensitive to touch or pressure or movement. In some embodiments, the nerve pain is sciatica or burning feet syndrome.

In one embodiment, the methods of the invention may be used to treat sports injuries such as bruising, corked muscles, muscle strains or muscle sprains, ligament damage, tendonitis or cartilage damage.

The methods of the invention also enhance healing of injuries to the skin such as cuts, rashes, abrasions, surgical incisions, ulcers and burns and enhance the healing of broken bones.

As used herein, the term “enhance healing” refers to decreasing the time taken for healing to occur. For example, recovery time for repair of broken bones or the repair of cuts, abrasions or surgical incisions is decreased by at least 10%, at least 20%, at least 30%, at least 40% or especially at least 50%.

In some embodiments the methods of the invention are used to relieve sore or stiff muscles. The sore or stiff muscles may be the result of injury or may be from doing strenuous exercise or a physical activity that the body or muscles are unaccustomed. In some embodiments the composition is applied to the skin in the vicinity of the sore, stiff or tired muscles.

In some embodiments, the methods of the invention may be used to treat animals in the racing industry such as horses or dogs. These animals are valuable and rely on good health and muscular tone for their livelihood. Furthermore, training and racing puts strain on their musculoskeletal system that may result in pain, muscle damage or muscle soreness or stiffness. The methods of the invention may be useful in treating pain, enhancing healing or relieving muscles soreness or stiffness in racing animals.

Accordingly, in one aspect of the invention, there is provided a method of treating or preventing pain or enhancing healing of an injury in a racing animal, comprising applying to the skin of the racing animal in the proximity of the injury, a topical medicinal formulation comprising 0.35 to 35% of at least one soluble salt of a biologically acceptable alkali metal or alkaline earth metal and a pharmaceutically acceptable carrier, wherein the formulation is in the form of a cream, lotion, ointment, liniment, gel, poultice, cold or hot compress, dressing or plaster cast.

By “about” is meant a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The invention will now be described with reference to the following examples which illustrate some preferred aspects of the present invention. However, it is to be understood that the particularity of the following description of the invention is not to supersede the generality of the preceding description of the invention.

### EXAMPLES

### Example 1

Fine grade natural sea salt 5.9% of composition (eg: Olsson's pacific fine salt) is mixed into sorbolene cream (94.1% of composition) until the salt is dissolved. The sorbolene cream used in this case was Woolworths Home Brand Sorbolene cream containing water, glycerine, sorbitol, stearic acid, mineral oil, cetearyl alcohol, Ceteth 20, triethanolamine, DMDM Hydantoin and tocopherol acetate.

### Example 2

A cream formulation of the present invention comprising 5.9% by weight of sea salt was tested on patients suffering pain. Each patient used the cream 2-3 times per day on the affected area while constant pain exists and the frequency reduced to once per day when pain eases.

Patients were asked to score their pain level on a scale of 1-10 before application and note the time taken for noticeable pain relief to occur.

The results are shown in Table 1.

TABLE 1

| | | | Initial pain | Time for | | % of relief |
|---|---|---|---|---|---|---|
| Patient | Problem | Position | score (1-10) | relief | | of pain |
| 1 | Padget's disease | degeneration of hip | 8/9 | 1 | week | 100% |
| | | and shoulder bones | | | | |
| 2 | Canal stenosis | back and leg | 8/9 (walking) | 2 | weeks | 80-90% |
| | | | 5 (at rest) | | | |
| 3 | Back ache¹ | back | 7 | 2 | months | 60% |
| 4 | Broken collarbone² | collarbone | 6 | instant | | 50% |
| 5 | Frozen shoulder | shoulder | 5 | 2 | weeks | 100% |
| 6 | Arthritic pain | both thumbs | 8 | 5 | minutes | 80% |
| 7 | Tennis elbow | elbow | 7 | overnight | | 60-70% |
| 8 | Arthritic pain³ | neck and shoulders | 8 | 3 | weeks | 80% |
| 9 | Arthritic pain | hip | 7 | ½ | hour | 100% |
| 10 | Bruising after strike | | 9 | 1 | hour | 100% |
| | with lawn bowl | | | | | |
| 11 | Osteoarthritis | knees | 8/9 | ½ | hour | 80-90% |
| 12 | Trauma⁴ | ankle | 9 | 2 | weeks | 100% |

| | | | | | |
|---|---|---|---|---|---|
| 13 | Dislocation | shoulder | 10 | instant | 30% |
| 14 | Corked⁵ | thighs | 8 | instant | 30% |
| 15 | Sprain | wrist | 6 | instant | 30% |
| 16 | Rolled ankle | ankle | 5 | instant | 30% |

¹Patient reported gradual relief after 1 week.
²Patient slept well after application of cream and healing of broken bone occurred in much shorter time than expected.
³Gradual relief at first.
⁴Pain reduced by 50% within 2 days. GP advised patient it would be 4 to 6 weeks before recovery of injury.
⁵Patient able to fully train for karate again after 2 days. Similar injuries had previously prevented full recovery from training for over 1 week.
⁶Patient was able to run on second day after injury.

### Example 3

A cream formulation of the present invention was prepared by mixing fine grade seal salt (Pacific Fine Sea Salt), magnesium chloride, ocean minerals and MSM (combined 9% weight of composition) until dissolved. Eucalyptus oil (2% w/w) and Arnica, Lantana Camara, Cuprum and hypericum extract (combined total of 5% w/w) were added and the cream thoroughly mixed to provide a homogenous formulation.

### Example 4

The cream formulation of Example 3 was tested on patients suffering pain, injury or specific nerve pain. Each patient used the cream 2-3 times per day on the affected area while constant pain was present and the frequency was reduced to once per day after the pain eased.

The results are shown in Tables 2 and 3. In the results shown in Table 2, the patients were asked to score their pain level on a scale of 1-10 before application and note the time taken for noticeable relief to occur. In Table 3, the patients provided comments but no numerical score.

TABLE 2

| | | Pain | | | | |
|---|---|---|---|---|---|---|
| | | Level out | Time for | | % of | |
| Patient: | Problem: | of 10 | relief: | | relief | Comment: |
| 17 | Paget's Disease | 8/9 | 1 | wk | 100 | Bone degeneration- |
| | | | | | | Hip and shoulder |
| 18 | Canal Stenosis | 8/9 when | 2 | wks | 80/90 | Back and leg |
| | | walking- | | | | |
| | | 5 other | | | | |
| | | times | | | | |
| 19 | Back ache | 7 | 2 | months | 60 | Gradual relief after |
| | | | | | | one week |
| 20 | Broken collarbone | 6 | Instant | | 50 | Slept well on first and |
| | | | | | | subsequent nights |
| 21 | Frozen shoulder | 5 | 2 | wks | 100 | Can now lift arm |
| | | | | | | above shoulder-no |
| | | | | | | pain |
| 22 | Arthritic pain | 8 | 5 | mins | 80 | Both thumbs |
| 23 | Arthritic pain | 7 | ½ | hour | 100 | Hip-off anti- |
| | | | | | | inflammatory |
| | | | | | | medicine |
| 24 | Struck by lawn bowl | 9 | 1 | hour | 100 | Constant reduction |
| | | | | | | from application |
| 25 | Osteoarthritis | 8/9 | ½ | hour | 80/90 | Both knees-off anti- |
| | | | | | | inflammatory |
| | | | | | | medicine |
| 26 | Severe trauma to ankle | 9 | 2 | wks | 100 | Pain reduced by 50% |

| | | | | | |
|---|---|---|---|---|---|
| | | | | | within two days- |
| | | | | | total recovery time |
| | | | | | estimated by GP |
| | | | | | halved |
| 27 | Neuropathy | 8 | Instant | 100 | Burning Feet |
| | | | | | Syndrome |
| 28 | Football Injury | 8 | Instant | 40 | Knee cartilage |
| | | | | | damage, strained hip |
| | | | | | and calf muscles |
| 29 | Re-dislocated shoulder | 10 | Instant | 30 | |
| | Corked thigh muscles | 8 | Instant | 30 | Full training after |
| | | | | | 2 days |
| | Sprained wrist | 6 | Instant | 30 | |
| | Rolled ankle | 6 | Instant | 30 | Running after 2 days |

TABLE 3

| Patient: | Problem: | Comment: |
|---|---|---|
| 30 | Back and hip pain | Significant improvement |
| 31 | Knee pain | Problem disappeared after a few applications |
| 32 | Hip pain-bone on bone | Relieves pain |
| 33 | Numerous injuries | Pain relieved-off anti-inflammatory drugs |
| | (TPI Army Veteran) | |
| 34 | Foot pain | Pain relieved as was the arthritic pain in hand used to |
| | | massage foot |
| 35 | Torn ligaments-inner | GP estimated 6 months repair time-used cream from forth |
| | thigh tendon | week-started running three weeks later without pain |
| 36 | Onset Muscle Soreness | Level significantly reduced on use as an after-workout |
| | | massage lotion |
| 37 | Onset Muscle Soreness | Level significantly reduced on use as an after-workout |
| | | massage lotion |
| 38 | Acupuncturist and | RSI injuries pain free almost immediately-injury time |
| | Remedial Therapist | from sprains, corked muscles and hamstring strains |
| | | reduced significantly |
| 39 | Muscle strain and knee | Pain relieved quickly allowing further training |
| | pain from swimming | |
| | training | |
| 40 | Arthritis in neck, vertebra | Protracted period of pain relief medication 2-3 times a |
| | out of line, neck and | day-after three weeks of nightly application totally off |
| | lower back pain | pain relief medication |
| 41 | Sports trainer at Aussie | Use on an extensive range of injuries to limit the intensity |
| | Rules Club | and severity of injuries. In particular the recovery from |
| | | cramps and corked muscles can occur in a quarter of |
| | | normal expected recovery time. |

### Example 5

A cream formulation containing

| Component | Percentage |
|---|---|
| Vegetable Sorbolene | 52% |
| Bioavailable oxygen | 25% |
| Bacteriostatic Pacific Sea Salt | 7.39% |
| Ethanol | 1.5% |
| Boswellia Concentrate powder (10-1) | 0.8% |
| Glucosamine hydrochloride | 2% |
| Eucalyptus oil | 2% |
| Arnica oil | 0.8% |
| Methyl Salicylate oil | 0.8% |
| Magnesium Chloride | 0.9% |
| Ocean Minerals (liquid) | 0.9% |
| Clove bud oil | 0.45% |
| Peppermint oil | 0.45% |
| Aloe Vera juice | 0.25% |
| Methyl Sulfonyl Methane | 0.25% |
| Winter greens oil | 1% |
| Black pepper oil | 1% |

The Boswellia concentrate powder was solubilized and then added, together with the other ingredients to sorbolene cream. The composition is then thoroughly mixed to provide a homogenous mixture.

1.-12. (canceled)

13. A topical medicinal formulation comprising 0.35 to 35% of at least one soluble salt of a biologically acceptable alkali metal or alkaline earth metal and a pharmaceutically acceptable carrier, wherein the formulation is in the form of a cream, lotion, ointment, liniment, gel, poultice, cold or hot compress, dressing or plaster cast, and wherein the at least one soluble salt is sea salt.

14. The topical medicinal formulation of claim 13, in the form of a cream, lotion or ointment.

15. The topical medicinal formulation of claim 13 wherein the soluble salt is present in an amount of 3% to 10%.

16. A method of treating or preventing pain comprising applying to the skin of a subject a topical medicinal formulation comprising 0.35 to 35% of at least one soluble salt of a biologically acceptable alkali metal or alkaline earth metal and a pharmaceutically acceptable carrier, wherein the formulation is in the form of a cream, lotion, ointment, liniment, gel, poultice, cold or hot compress, dressing or plaster cast, and wherein the at least one soluble salt is sea salt.

17. A method of enhancing healing of an injury in a subject comprising applying to the skin of the subject in the proximity of the injury a topical medicinal formulation comprising 0.35 to 35% of at least one soluble salt of a biologically acceptable alkali metal or alkaline earth metal and a pharmaceutically acceptable carrier, wherein the formulation is in the form of a cream, lotion, ointment, liniment, gel, poultice, cold or hot compress, dressing or plaster cast, and wherein the at least one soluble salt is sea salt.

18. The method according to claim 16 wherein the pain is musculoskeletal pain, nerve pain, pain caused by trauma, or headaches.

19. The method according to claim 17 wherein the subject is a human or a racing animal.

20. A method of relieving muscle soreness or stiffness or tired muscles comprising applying to the skin of a subject a topical medicinal formulation comprising 0.35 to 35% of at least one soluble salt of a biologically acceptable alkali metal or alkaline earth metal and a pharmaceutically acceptable carrier, wherein the formulation is in the form of a cream, lotion, ointment, liniment, gel, poultice, cold or hot compress, dressing or plaster cast, and wherein the at least one soluble salt is sea salt.

21. A method of treating or preventing ulcers comprising applying to the skin of a subject a topical medicinal formulation comprising 0.35 to 35% of at least one soluble salt of a biologically acceptable alkali metal or alkaline earth metal and a pharmaceutically acceptable carrier, wherein the formulation is in the form of a cream, lotion, ointment, liniment, gel, poultice, cold or hot compress, dressing or plaster cast, and wherein the at least one soluble salt is sea salt.

22. The method according to claim 18 wherein the subject is a human or racing animal.
